(19) United States
(12) Patent Application Publication
(10) Pub. No.: US 2023/0301551 A1
(43) Pub. Date: Sep. 28, 2023

(54) INTERACTIVE MODULAR HEARING SERVICES SOUNDPROOF KIOSK

(71) Applicant: Sonova AG, Stäfa (CH)

(72) Inventors: Sven Davies, Naperville, IL (US); Pauline Dinnauer, Mentone, CA (US)

(21) Appl. No.: 17/656,484

(22) Filed: Mar. 25, 2022

(51) Int. Cl. A61B 5/12; G06F 3/0482; G06F 3/14; H04R 25/00; H04R 1/02; A61B 5/00

(52) U.S. Cl. CPC A61B 5/123; G06F 3/0482; G06F 3/14; H04R 25/70; H04R 1/025; H04R 25/558; H04R 25/554; A61B 5/002; A61B 5/0022; H04R 2225/55

(57) ABSTRACT

An exemplary interactive hearing services kiosk includes one or more walls configured to define a space; a first door providing access to the space; one or more media devices; one or more user display screens, at least one of the one or more user display screens is located on the outside of at least one of the one or more walls of the kiosk so as to define an outside display screen; and a controller. The controller may be configured to display, via the outside display screen, one or more prompts that are associated with one or more hearing services and receive input that is responsive to the one or more prompts. The input may include at least one response via the one or more media devices. The controller may be configured to validate the input and generate one or more recommendations based on a validated input.

### TECHNICAL FIELD

The present disclosure relates to soundproof kiosks, and more particularly, to systems and methods for interactive modular hearing services soundproof kiosks.

### BACKGROUND

Health concerns and convenience have motivated many people to look at remote health services. Numerous challenges exist in identifying qualified professionals who can competently provide proper patient care. In addition, a regular retail location to offer such services may take about six months or more to build out, and there are limitations in staffing and interacting with prospective and existing users. These and other deficiencies exist.

### BRIEF SUMMARY

Embodiments of the present disclosure provide an interactive hearing services kiosk. The kiosk may include one or more walls configured to define a space; a first door providing access to the space; one or more media devices; one or more user display screens at least one of the one or more user display screens is located on the outside of at least one of the one or more walls of the kiosk so as to define an outside display screen; and a controller. The controller may be configured to display, via the one or more user display screens, a first set of one or more prompts that are associated with one or more hearing services. The controller may be configured to receive a first input that is responsive to the first set of one or more prompts, the first input including at least one response via the one or more media devices. The controller may be configured to validate the first input. The controller may be configured to generate one or more recommendations based on a validated first input.

Embodiments of the present disclosure provide a method including the step of generating, by a controller, a first set of one or more prompts that are associated with one or more hearing services. The method may include instructing, by the controller, an outside display screen to display the first set of one or more prompts. The method may include receiving, by the controller, a first input that is responsive to the one or more prompts, the first input including at least one response via one or more media devices. The method may include validating, by the controller, the first input. The method may include generating, by the controller, one or more recommendations based on a validated first input.

Embodiments of the present disclosure provide a computer readable storage medium comprising computer program code instructions, being executable by a computer, for: generating one or more prompts that are associated with one or more hearing services; displaying the one or more prompts; receiving input that is responsive to the one or more prompts, the input including at least one response via one or more media devices; validating the input; and generating one or more recommendations based on a validated input.

### BRIEF DESCRIPTION OF THE DRAWINGS

FIG. 1 illustrates an interactive modular hearing services soundproof kiosk according to an example embodiment.

FIG. 2 illustrates an interactive modular hearing services soundproof kiosk according to an example embodiment.

FIG. 3 illustrates an interactive modular hearing services soundproof kiosk according to an example embodiment.

FIG. 4 illustrates an interactive modular hearing services soundproof kiosk according to an example embodiment.

FIG. 5 illustrates a plan view of an interactive modular hearing services soundproof kiosk.

FIG. 6 illustrates a method of initial interaction with a user using the interactive modular hearing services soundproof kiosk.

FIG. 7 illustrates a method of interaction with a user using the interactive modular hearing services soundproof kiosk according to an example embodiment.

FIG. 8 illustrates a method of interaction with a user using the interactive modular hearing services soundproof kiosk according to an example embodiment.

FIG. 9 illustrates a method of interaction with a user using the interactive modular hearing services soundproof kiosk according to an example embodiment.

FIG. 10 illustrates a method of interaction with a user using the interactive modular hearing services soundproof kiosk according to an example embodiment.

FIG. 11 illustrates a method of interaction with a user using the interactive modular hearing services soundproof kiosk according to an example embodiment.

FIG. 12 illustrates a method of interaction with a user using the interactive modular hearing services soundproof kiosk according to an example embodiment.

FIG. 13 illustrates a schematic diagram according to an example embodiment.

### DETAILED DESCRIPTION

The following description of embodiments provides non-limiting representative examples referencing numerals to particularly describe features and teachings of different aspects of the invention. The embodiments described should be recognized as capable of implementation separately, or in combination, with other embodiments from the description of the embodiments. A person of ordinary skill in the art reviewing the description of embodiments should be able to learn and understand the different described aspects of the invention. The description of embodiments should facilitate understanding of the invention to such an extent that other implementations, not specifically covered but within the knowledge of a person of skill in the art having read the description of embodiments, would be understood to be consistent with an application of the invention.

The systems and methods disclosed herein provide an interactive programmable platform for prospective and existing patients for dynamic interactions that facilitate enablement and improved efficiency of remote health services, including onsite programmable hearing devices based on real-time hearing evaluations and simulating acoustic environments. In addition to achieving the benefit of interacting with a remote audiologist, instead of having one on site, each audiologist can comfortably support a plurality of kiosks instead of one-to-one for a retail location. Through the interactive platform, the programmable kiosk enables remote care for users seeking a hearing health solution. As further disclosed herein, the interactive kiosk is configured to provide a full range of hearing services by onsite and/or remote audiology professionals. With only a few days to assemble the kiosk, the kiosks may be conveniently placed in numerous areas, such as hospital lobbies, malls, convention centers, airports, retirement communities, clinics, and the like.

The systems and methods disclosed herein also allow for interactive programmable kiosks with integrated media devices to provide an improved interactive user experience to help facilitate the improved efficiency of remote health services. In particular, this may be achieved by allowing a user to independently interact and learn about and act upon identification of hearing issues through an initial screener evaluation outside the kiosk followed by another detailed evaluation inside the kiosk that is configured to utilize the real-time input from integrated media devices, such as bone transducer and video otoscopy. Programmable hearing devices may also be the consequence of the evaluations, which may also be adjusted in real-time for fitting and troubleshooting. Variations of the systems and methods disclosed herein may include different size kiosks, or varying marketing technology and messaging that may be placed on the outside of the kiosk to improve the user experience, and taking minimal sources of input via integrated media devices of the kiosk to provide for real-time programmable and troubleshooting hearing devices. By way of example, a hearing aid may be fitted such as is disclosed by in U.S. Pat. No. 9,774,965, the entire disclosure of which is hereby incorporated by reference. In some examples, the kiosk may be controlled, by a controller, through assistance of an individual, such as a licensed healthcare professional. In other examples, the kiosk may comprise a self-service kiosk that is controlled by the controller. In particular, it may be preferable for the kiosk to operate in an attended manner. In other examples, it may be preferable for the kiosk to operate in an automated manner. Yet further in other examples, it may be preferable for the kiosk to operate in a combination thereof.

As referred to herein, hearing devices may be used to improve the hearing capability or communication capability of a user, for instance by compensating a hearing loss of a hearing-impaired user, in which case the hearing device may be referred to as a hearing instrument such as a hearing aid, or hearing prosthesis. A hearing device may also be configured to output sound based on an audio signal which may be communicated to, by a wire or wirelessly, the hearing device. A hearing device may also be configured to reproduce a sound in a user's ear canal detected by a microphone. The reproduced sound may be amplified to account for a hearing loss, such as in a hearing instrument, or may be output without accounting for a hearing loss, for instance to provide for a faithful reproduction of detected ambient sound and/or to add sound features of an augmented reality in the reproduced ambient sound, such as in a hearable. A hearing device may also provide for a situational enhancement of an acoustic scene, e.g. beamforming and/or active noise cancelling (ANC), with or without amplification of the reproduced sound. A hearing device may also be implemented as a hearing protection device, such as an earplug, configured to protect the user's hearing. Different types of hearing devices may be configured to be worn at and/or in an ear may include, without limitation, earbuds, earphones, hearables, and hearing instruments such as receiver-in-the-canal (RIC) hearing aids, behind-the-ear (BTE) hearing aids, in-the-ear (ITE) hearing aids, invisible-in-the-canal (IIC) hearing aids, completely-in-the-canal (CIC) hearing aids, cochlear implant systems configured to provide electrical stimulation representative of audio content to a user, a bimodal hearing system configured to provide both amplification and electrical stimulation representative of audio content to a user, or any other suitable hearing prostheses. In some examples, a hearing system may comprise two hearing devices configured to be worn at different ears of the user may be referred to as a binaural hearing device. Without limitation, a user of the kiosk may refer to a patient or a client or a proxy thereof.

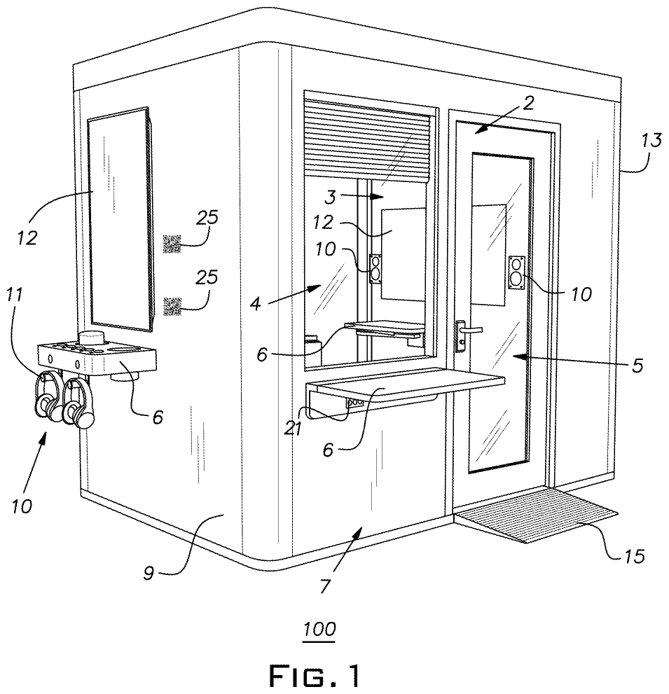

FIG. 1 illustrates an interactive modular hearing services soundproof kiosk, such as an interactive hearing services soundproof kiosk 100. The kiosk 100 may include a first door 2; a first window 4; a first plurality of shelves 6; a bench seat 8; one or more integrated media devices 10; and one or more user display screens 12. In addition, the kiosk 100, in particular a control system of it (not shown), may be programmable and configured to perform and control, via a control system such as control system 1300 including a controller such as controller 1302, any component(s) of kiosk 100, as further disclosed herein. As further explained below, the control system of the kiosk 100 may be configured to control the one or more integrated media devices 10 and/or the one or more user display screens 12. Although FIG. 1 illustrates single instances of the components, kiosk 100 may include any number of components.

The kiosk 100 may include one or more walls configured to define a space, and a first door 2 providing access to the space. By way of example, the space may comprise a room. In some examples, the one or more walls may comprise a single wall. In other examples, the one or more walls may comprise two or more walls. The first door 2 may include a second window 5. The kiosk 100 may include a first window 4. The first window 4 may be disposed adjacent the first door 2. For example, the first window 4 may comprise a first distance in the x-direction and a first distance in the y-direction. The second window 5 may comprise a second distance in the x-direction and a second distance in the y-direction. In some examples, the second distance in the y-direction may be greater than the first distance in the y-direction, and the first distance x-direction may be greater than the second distance in the x-direction. In some examples, the first window 4 may comprise same or different distances in x-direction and/or y-direction as that of the second window 5. It is understood that these distances are exemplary and not intended to be limiting in any manner. The first window 4 may be disposed towards a corner of the kiosk 100.

The kiosk 100 may include a first plurality of shelves 6. At least a first shelf 6 selected from the first plurality of shelves may be connected to a first interior surface 3 of the kiosk 100. The first shelf 6 may be arranged opposite to the bench seat 8 (not shown). A second shelf 6 selected from the first plurality of shelves may be connected to a first exterior surface 9. A third shelf 6 selected from the first plurality of shelves may be connected to a second exterior surface 7. In some examples, the first shelf 6 selected from the plurality of shelves comprises a rigid shelf such that it is fixed and non-adjustable. In some examples, the second shelf 6 selected from the plurality of shelves comprises a rigid shelf such that it is fixed and non-adjustable. In some examples, the third shelf 6 selected from the plurality of shelves comprises a non-rigid shelf such that it is adjustable and foldable. In other examples, any of the shelves 6, including the first, second, and third shelf, may be rigid or non-rigid. In addition, any of the shelves 6 may be of same or different sizes, shapes, and/or areas. The shelves 6 may be configured to extend outwards. For example, the first shelf 6 may be configured to protrude outwards from the first interior surface 3 of the kiosk 100. The second shelf 6 may be configured to protrude outwards from the first exterior surface 9 of the kiosk 100. In some examples, the second shelf 6 may comprise one or more containers configured to hold cleaning materials, including but not limited to sanitizing wipes. Without limitation, the sanitizing wipes of second shelf 6 may be accessed for use to clean or wipe any portion and component of the kiosk 100, including user display screens 12, any number and/or type of one or more integrated media devices 10, and/or any combination thereof. The third shelf 6 may operate a self-securing mechanism, such that it may fold downwards so as to be disposed in a direction parallel to the second exterior surface 7 of the kiosk 100 so as to lock, and thereby avoid exposure of a plurality of ports 21 of the kiosk 100. A key may then be used to unlock the third shelf 6. In some examples, the plurality of ports may be configured to provide access to control and operations of the kiosk 100. In some examples, the plurality of ports may refer to the plurality of ports 21, as described below. In other examples, the plurality of ports may be different than the plurality of ports 21. The third shelf 6 may be configured to project outwards away from the second exterior surface 7 of the kiosk 100. In some examples, any of the first, second, and third shelves 6 may take on a different or same form as the remaining shelves and may be configured accordingly. Thus, it is understood that the first, second, and third shelves 6 are not limited to only the descriptions set forth above. For example, any of the shelves 6 may comprise the one or more containers and/or any of the shelves may be configured to operate the self-securing mechanism.

The kiosk 100 may include one or more integrated media devices 10. While only headphones 10 and speakers 10 are depicted, any other types of integrated media devices may be included. In addition, each of the integrated media devices 10 may be integrated through a network (not shown) for connectivity, such as the Internet. Accordingly, in some examples, the kiosk 100 may include, in addition to the headphones 10 and speakers 10, one or more additional media devices 10, such as one or more additional integrated media devices. Without limitation and by way of example, the one or more integrated media devices 10 and/or the one or more additional integrated media devices 10 may include one or more selected from the group of a web camera, an audiometer, a video otoscope, a bone conduction transducer, a device configured for real-ear verification, an operator headphone, an operator microphone, a patient headphone, a patient microphone, an electronic signature pad, a patient response button, a push button interface, or a speaker. In some examples, any of the one or more integrated media devices 10 may be controlled by the control system (not shown). In some examples, speakers 10 may be part of one or more speakers that are configured to simulate one or more acoustic environments for a predetermined time duration. The one or more acoustic environments may be associated with a hearing evaluation to program, by the control system, without limitation, a hearing device, a hearing instrument, a hearing aid, a hearing prosthesis, a hearing protection device, and/or a hearing system, as previously explained above. Information for creating acoustic environments can be stored in memory at the kiosk 100, such as in memory of a control system, or communicated from a remote location to the kiosk 100.

The kiosk 100 may include one or more user display screens 12 that are controlled by the control system (not shown). In some examples, at least one of the user display screens 12 may be located on the first interior surface 3 of the kiosk 100, and may be referred to as the first user display screen. For example, the first user display screen 12 may be a user display screen that is internal to the kiosk 100. For example, at least one of the user display screens 12 may be disposed above the first shelf 6 and between speakers 10. In some examples, at least one of the user display screens 12 may be located on the first exterior surface 9 of the kiosk 100, and may be referred to as the second user display screen. For example, the second user display screen 12 may be a user display screen external to the kiosk 100. For example, at least one of the user display screens 12 may be disposed above the second shelf 6. In some examples the first user display screen 12 may comprise a different width than that of the second user display screen 12. The one or more user display screens 12 may also be configured, by the control system, to connect to the network (not shown). The first user display screen may have the same size as the second user display screen. In other examples, the first user display screen may be a different size from the second user display screen. In addition, the first user display screen may be oriented in a same direction, such as horizontally or vertically, as the second user display screen. In other examples, the first user display screen may be oriented in a different direction, such as horizontally, when compared to the second user display screen, such as vertically, or vice versa. In other examples, the first or the second user display screen 12 may be configured as a stand-alone screen. In other examples, the first user display screen 12, such as the user display screen that is located on the first interior surface 3 of the kiosk 100, may be configured as a stand-alone screen. In some other examples, the second user display screen 12, such as the user display screen that is located on the first exterior surface 9 of the kiosk 100, may be configured as a stand-alone screen. Accordingly, the first user display screen may be located inside the kiosk 100 and the second user display screen may be located outside the kiosk 100. Without limitation, the kiosk 100 may include one or more additional user display screens 12, such as one or more additional first user display screens and/or more additional second user display screens. Thus, the kiosk 100 may include any number of internal and/or external user display screens.

The one or more user display screens 12 may be configured to display, by the control system, one or more prompts that are associated with one or more health services. Without limitation, the one or more health services may include hearing services, such as hearing evaluations, video otoscopy, programming any number of a hearing device, a hearing instrument, a hearing aid, a hearing prosthesis, a hearing protection device, and/or a hearing system in real-time or substantially in real-time including real ear verification on a hearing device, a hearing instrument, a hearing aid, a hearing prosthesis, a hearing protection device, and/or a hearing system; hearing appointment scheduling; troubleshooting a programmed or non-programmed hearing device, a hearing instrument, a hearing aid, a hearing prosthesis, a hearing protection device, and/or a hearing system in real-time or substantially in real-time; facilitating purchase transaction of any number of new and/or recently programmed hearing device, a hearing instrument, a hearing aid, a hearing prosthesis, a hearing protection device, and/or a hearing system; providing audio and/or video conference capabilities with a remote health-care professional; simulating one or more acoustic environments for a predetermined time duration; and/or any combination thereof.

In some examples, the one or more acoustic environments may be generated to create a predetermined listening environment. The predetermined listening environment may facilitate a user experience to depict how improvement of their hearing will be with the programmed one or more of a hearing device, a hearing instrument, a hearing aid, a hearing prosthesis, a hearing protection device, and/or a hearing system. In addition, the one or more acoustic environments may operate in tandem with one or more lights, such as lights 23, of the kiosk 100 to create the predetermined listening environment. Without limitation, the predetermined listening environment may take into account adjustment by the kiosk 100 of a brightness value of any of the one or more lights 23, such as being dimmed as if an individual were in a concert hall environment; or if an individual were in an outdoor environment walking down a path, where the one or more lights brighten as the individual comes out into the sun, or the one or more lights dim as the individual approaches a shady area. Once one or more of the hearing device, the hearing instrument, the hearing aid, the hearing prosthesis, the hearing protection device, and/or the hearing system are programmed and fitted by the control system, the user would wear them and experience the different acoustic environments that are simulated, including but not limited to hearing every note during a concert; hearing the birds; or the wind blowing through trees while walking outside. At least one of the one or more user display screens 12 may, by the control system, be configured to prompt selection of the one or more acoustic environments via touch screen on a menu. Alternatively, the one or more acoustic environments may be selected via audible input, such as where the user speaks to select the predetermined listening environment.

The one or more user display screens 12 may be configured, for example by the control system, to receive input that is responsive to the one or more prompts. The input may include at least one response via the one or more integrated media devices 10. The one or more user display screens 12 may be configured to, by the control system, validate the input. The one or more user display screens 12 may be configured to, by the control system, generate one or more recommendations based on the validated input. The one or more user display screens 12 may be configured to, by the control system, program one or more of the hearing device, the hearing instrument, the hearing aid, the hearing prosthesis, the hearing protection device, and/or the hearing system based on the one or more recommendations.

At least one of the user display screens selected from the one or more user display screens 12 may be configured to, by the control system, prompt a user for a first input responsive to a first evaluation. For example, the first evaluation may comprise a screener hearing evaluation that is displayed on the second user display screen that is located outside the kiosk 100, such as the user display screen that is configured to be located on the first exterior surface 9 of the kiosk 100. In some examples, the second user display screen may be referred to as the outside or exterior display screen. The first evaluation may comprise a first limited or first fixed number of first set of one or more prompts that contain one or more first queries, in which the first input may be provided by one or more of the one or more integrated media devices 10 as one or more respective first inputs. By way of example, the first evaluation may serve as a non-clinical test that determines the possibility of whether or not hearing loss exists. In some examples, the resulting outcome or possibility determined from the first evaluation may be used to determine whether or not a second evaluation should be performed. In addition, the first evaluation may also serve as an informational resource to the user by informing them about hearing loss.

At least one of the other user display screens, such as the first user display screen that is configured to be located on the first interior surface 3 of the kiosk 100, selected from the one or more user display screens 12 may be configured to, by the control system, prompt the user for a second input responsive to the second evaluation. In some examples, the first user display screen may be referred to as the inside or interior display screen. The second user display screen, or the inside or interior display screen, may be configured, by the control system, perform the second evaluation and perform the fitting of one or more of the hearing device, the hearing instrument, the hearing aid, the hearing prosthesis, the hearing protection device, and/or the hearing system. The second evaluation may comprise a second limited or second fixed number of second set of one or more prompts that contain one or more second queries, in which the second input may be provided by one or more of the one or more integrated media devices 10 as one or more respective second inputs. By way of example, the second evaluation may serve as a clinical test, and may be performed depending on the outcome of the first evaluation. Without limitation, the second evaluation may encompass a more formal, clinical evaluation (as opposed to the first or non-clinical evaluation by the second user display screen that is located on the first exterior surface 9 of the kiosk 100), using any number of the one or more integrated media devices 10, including but not limited to a calibrated audiometer, a device configured for real-ear verification, video otoscopy, air and bone conduction tests, and/or any combination thereof.

Each of the first and second inputs may be responsive to the first and second queries, respectively, and provided, by the control system, to the respective user display screens 12 in an interactive manner so as to be received in real-time for validation. For example, the first input may be provided, responsive to the first query, to the second user display screen 12, and the second input may be provided, responsive to the second query, to the first user display screen 12. In some examples, the first input may be responsive to the first set of one or more prompts and provided to the second user display screen 12, and the second input may be responsive to the second set of one or more prompts and provided to the first user display screen 12. It is understood that such sequence of providing the first input followed by the second input is not limiting: for example, the second input may be provided first followed by the first input; only the first input may be provided; only the second input may be provided; and/or any combination of the first and second inputs may be provided, as disclosed herein. The first and the second inputs may be provided via the one or more integrated media devices 10, via audio input, via video input, via touch screen input, via non-touch screen input, and/or any combination thereof. For example, at least one of the evaluations, such as the second evaluation, may include one or more queries than that belonging to the other evaluation, such as the first evaluation. In some examples, the user may refer to the same user. In other examples, the user may refer to a different user.

Upon receipt of the input, the one or more user display screens 12 may be each configured to process, by the control system, the first and second inputs to yield separate outcomes. As discussed above, the first evaluation may serve as a non-clinical test that determines a first outcome of the possibility of whether or not hearing loss exists, whereas the second evaluation may serve as a clinical test that determines a second outcome of the nature of the hearing loss at specific frequencies. After completion of the second evaluation and the hearing device, the hearing instrument, the hearing aid, the hearing prosthesis, the hearing protection device, and/or the hearing system are fitted for the hearing loss based on the second outcome, a device incorporating (or configured to incorporate) real-ear verification may be used to verify the fitting of the hearing device, the hearing instrument, the hearing aid, the hearing prosthesis, the hearing protection device, and/or the hearing system that are unique to the subject's hearing loss and ear canals. After the first and second inputs have been validated, the one or more user display screens 12 may be each configured to, by the control system, generate one or more recommendations based on the validated input. For example, a first set of recommendations may be generated, by the control system, based on validated first input. A second set of recommendations may be generated, by the control system, based on validated second input. In some examples, each of these recommendations may include at least one selected from the group of a particular type of one or more of the hearing device, the hearing instrument, the hearing aid, the hearing prosthesis, the hearing protection device, and/or the hearing system, a specified parameter(s) for adjustment associated with the one or more of the hearing device, the hearing instrument, the hearing aid, the hearing prosthesis, the hearing protection device, and/or the hearing system, a programming time of the one or more of the hearing device, the hearing instrument, the hearing aid, the hearing prosthesis, the hearing protection device, and/or the hearing system with or without the specified parameter(s), or a recommendation for further consultation. By way of example, the one or more recommendations may include one or more hearing device configuration parameters. In some examples, the control system (not shown), but as further explained below, may be configured to automatically program a hearing device, a hearing instrument, a hearing aid, the hearing prosthesis, a hearing protection device, and/or a hearing system from a location remote from the kiosk 100 with the one or more hearing device configuration parameters that are identified in the one or more recommendations. In other examples, the control system (not shown), but as further explained below, may be configured to manually program the hearing device, the hearing instrument, the hearing aid, the hearing prosthesis, the hearing protection device, and/or the hearing system with the one or more hearing device configuration parameters that are identified in the one or more recommendations. By way of example, the one or more hearing device configuration parameters may include a specific frequency gain in one or more channels available for the hearing device, the hearing instrument, the hearing aid, the hearing prosthesis, the hearing protection device, and/or the hearing system; feedback adjustments; a plurality of application programs configured to support improved hearing in noisy environments, such as restaurants, bars, airports, schools, churches, cafes, libraries, and other venues; and controls to support tinnitus management as available on the hearing device, the hearing instrument, the hearing aid, the hearing prosthesis, the hearing protection device, and/or the hearing system. By way of example, any of the one or more user display screens 12 may be configured to, by the control system, display a plurality of hearing evaluations, receive the input from a user that is responsive to the plurality of hearing evaluations, validate the input, and configure one or more of the hearing device, the hearing instrument, the hearing aid, the hearing prosthesis, the hearing protection device, and/or the hearing system based on the input. For example, the one or more hearing device, the hearing instrument, the hearing aid, the hearing prosthesis, the hearing protection device, and/or the hearing system may be programmed and fitted in real-time based on real-ear verification.

In some examples, any of the one or more user display screens 12 may be configured to, by the control system, detect one or more of the hearing device, the hearing instrument, the hearing aid, the hearing prosthesis, the hearing protection device, and/or the hearing system via a wired connection, a wireless connection, or any combination thereof. For example, the one or more user display screens 12 may be configured to, by the control system, establish communication for detection and identification of the of the hearing device, the hearing instrument, the hearing aid, the hearing prosthesis, the hearing protection device, and/or the hearing system via at least one selected from the group of near field communication (NFC), radio frequency identification (RFID), Bluetooth®, or Wi-Fi®. The hearing device, the hearing instrument, the hearing aid, the hearing prosthesis, the hearing protection device, and/or the hearing system may be a part of the one or more integrated media devices 10. In other examples, the of the hearing device, the hearing instrument, the hearing aid, the hearing prosthesis, the hearing protection device, and/or the hearing system may not be a part of the one or more integrated media devices 10.

The one or more user display screens 12 may be configured to, by the control system, program one or more of the hearing device, the hearing instrument, the hearing aid, the hearing prosthesis, the hearing protection device, and/or the hearing system based on the one or more recommendations. For example, any of the one or more user display screens 12 may be configured to, by the control system, securely connect to a remote system (not shown) that is associated with a health professional, and after establishing connection with the remote system, be configured to then, by the control system, program one or more of the hearing device, the hearing instrument, the hearing aid, the hearing prosthesis, the hearing protection device, and/or the hearing system based on received input responsive to a plurality of hearing evaluations. In some examples, only the inside display screen may be configured, by the control system, to program one or more of the hearing device, the hearing instrument, the hearing aid, the hearing prosthesis, the hearing protection device, and/or the hearing system. Once connection with the one or more user display screens 12 to the remote system has been established, the one or more of the hearing device, the hearing instrument, the hearing aid, the hearing prosthesis, the hearing protection device, and/or the hearing system may be configured to, by the control system, be programmed, including troubleshooting, in real-time. By way of example, troubleshooting may refer to controlled adjustment, such as by the control system of kiosk 100, of any of the one or more hearing device configuration parameters for one or more of the hearing device, the hearing instrument, the hearing aid, the hearing prosthesis, the hearing protection device, and/or the hearing system.

The kiosk 100 may comprise a plurality of hangers 11. For example, a first hanger selected from the plurality of hangers 11 may be connected to the second shelf selected from the first plurality of shelves 6 that may be connected to the first exterior surface 9, and a second hanger selected from the plurality of hangers 11 may be connected to a surface inside the kiosk 100, such as the first interior surface 3.

The kiosk may comprise a storage room 13. For example, the storage room 13 may be partitioned from the first door 2, and comprise a second plurality of shelves 14, a second door 16, and a third door 18.

The kiosk 100 may comprise a ramp 15. For example, the ramp 15 may be configured to incline at a predetermined angle relative to a first axis. In some examples, the angle may include a range from two degrees to five degrees, such as four degrees, that is relative to an axis parallel to the ground. The ramp 15 may be connected to the first door 2.

The kiosk 100 may comprise a plurality of fans 17 and a plurality of vents 19 so as to provide air circulation within the kiosk 100. The kiosk may comprise a plurality of baffle panels 20; a plurality of ports 21; carpet tile 22; a plurality of lights 23; and a plurality of arm rests 24. By way of example and without limitation, the plurality of ports 21 may include one or more selected from the group of an electrical outlet or a universal serial bus port. Any other type of port configured for power or device connection may also be included with the plurality of ports 21.

The kiosk 100 may comprise a terminal (not shown), such as a contactless or a contact payment system, configured to process, by the control system, a transaction associated with at least one of a trial or a purchase of one or more of the hearing device, the hearing instrument, the hearing aid, the hearing prosthesis, the hearing protection device, and/or the hearing system that are programmed according to received input responsive to one or more hearing evaluations at one or more of the user display screens 12.

The user may interact with the kiosk 100 via the one or more user display screens 12 that may be controlled by the control system. Without limitation, the interaction may take place by a device (including but not limited to a mobile device, a tablet, or the like) that includes a processor and a memory comprising an application that includes instructions for execution therein. In some examples, the device may interact with the kiosk 100 by scanning one or more QR codes 25 that are displayed on a surface of the kiosk 100. Without limitation as to location or placement, the one or more QR codes 25 may be displayed anywhere, including but not limited to any surface, component, or part of the kiosk 100, such as the exterior surface 9 of the kiosk 100. Additionally or alternatively, and by way of example, the one or more QR codes 25 may be displayed on the first interior surface 3, and/or the second interior surface 7 of the kiosk 100, and/or on the second user display screen 12 (not shown). Responsive to scanning the one or more QR codes 25 by the device, or any other device configured to read the one or more QR codes 25, the user may be prompted to click a link, such as launch a website and/or input login credentials and/or launch an application. In addition, scanning of the one or more QR codes 25 may be configured such that identification is possible, in particular such that it helps identify: i) which kiosk 100, amongst a plurality of kiosks 100, is available; and ii) which of user display screens 12, such as the first and second user display screens 12 of each the plurality of kiosks 100 are interactive and/or available.

In addition, the user may interact with the kiosk 100 via biometric input (including but not limited to a fingerprint, eye scan, voice recognition, and/or any combination thereof) and/or haptic input (including but not limited to any type of touch input) that is received by any of the one or more user display screens 12. In some examples, the biometric input and/or haptic input may be responsive to one or more requests from the kiosk 100 requiring input of a security code in order to gain access to the kiosk 100 and/or enable functionality of the kiosk 100, including but not limited to interacting with any of the components of the kiosk 100, such as the one or more user display screens 12. The security code may be associated with a manual and/or digital lock that is integrated with the kiosk 100. The security code may be in addition to, or in alternative to, the input login credentials. Without limitation, the security code may include a predetermined sequence of numbers, symbols, characters, and/or any combination thereof. Thus, the kiosk 100 may be configured to authenticate the inputted security code, the biometric input, and/or the haptic input.

The kiosk 100 may be further configured to include built-in internet, patient intake, hearing services self-serve, hearing services manned, audiology testing, payment processing, fittings of one or more of the hearing device, the hearing instrument, the hearing aid, the hearing prosthesis, the hearing protection device, and/or the hearing system, motion activations via one or more sensors to trigger audio and/or video conferencing with a remote health-care professional, appointment scheduling. In addition, the kiosk 100 may further configured to be soundproof, include security protocols for access and use permissions, handicap (such as wheelchair) accessible including but not limited to the ramp 15, easily shippable and assembled. As referred to herein, the term “soundproof” may include the definition set forth under American National Standards Institute (ANSI) of maximum permissible ambient noise levels (MPANLs) for audiometric threshold assessment, which are specified in ANSI S3.1-1991. It is further understood that the kiosk 100 may be minimally soundproof, such that it meets at least the ANSI standard, and that in some examples, the kiosk 100 may be superior to the ANSI standard. Moreover, the kiosk 100 may be configured to implement any of the following: prospective and/or existing user(s) looking for hearing health information; prospective and/or existing user(s) wishing to evaluate and test hearing; prospective and/or existing user(s) wishing to try or purchase one or more of the hearing device, the hearing instrument, the hearing aid, the hearing prosthesis, the hearing protection device, and/or the hearing system; prospective and/or existing user(s) seeking general support with one or more of the hearing device, the hearing instrument, the hearing aid, the hearing prosthesis, the hearing protection device, and/or the hearing system; prospective and/or existing user(s) seeking remote fitting and/or adjustment of one or more of the hearing device, the hearing instrument, the hearing aid, the hearing prosthesis, the hearing protection device, and/or the hearing system existing user(s) seeking new one or more of the hearing device, the hearing instrument, the hearing aid, the hearing prosthesis, the hearing protection device, and/or the hearing system; prospective and/or existing user(s) seeking an in-person or audio and/or video conference appointment with health-care professional (HCP); and prospective and/or existing user(s) working with onside health-care professional.

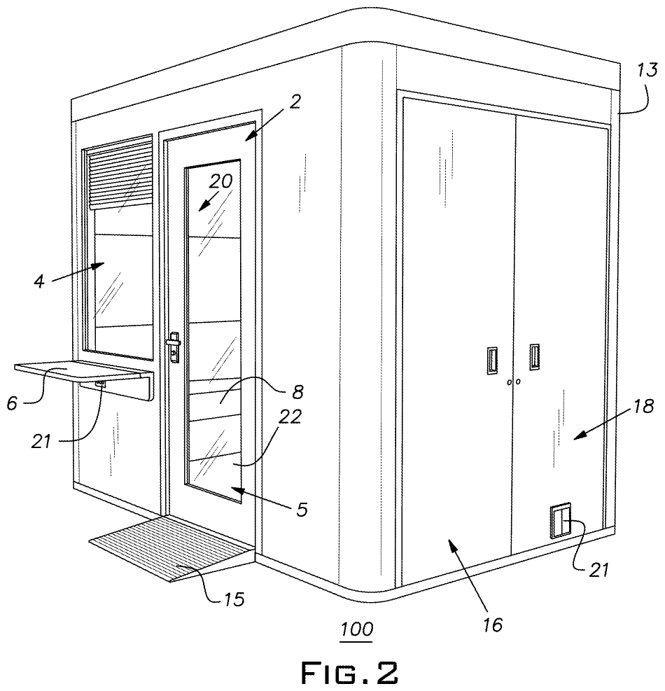

FIG. 2 illustrates an interactive modular hearing services soundproof kiosk 100. Although FIG. 2 illustrates single instances of the components, kiosk 100 may include any number of components. The kiosk 100 of FIG. 2 may reference and incorporate any components of kiosk of FIG. 1. As shown in FIG. 2, the kiosk 100 includes a seat bench 8 and the storage room 13. The storage room 13 includes doors 16, 18 that are illustrated as in the closed position.

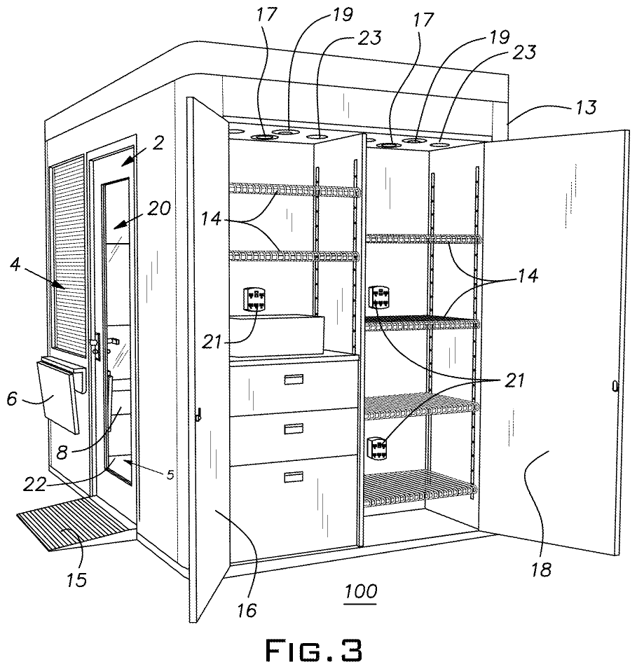

FIG. 3 illustrates an interactive modular hearing services soundproof kiosk 100. Although FIG. 3 illustrates single instances of the components, kiosk 100 may include any number of components. The kiosk 100 of FIG. 3 may reference and incorporate any components of kiosk of FIGS. 1-2. As shown in FIG. 3, the kiosk 100 includes the contents revealed behind the doors 16, 18 of storage room 13, such as second plurality of shelves 14 and plurality of ports 21. In addition, the plurality of fans 17, plurality of vents 19, and plurality of lights 23 may be disposed in an upper portion above the storage room 13. It is understood that the plurality of fans 17, plurality of vents 19, and plurality of lights 23 are not limited to such arrangements, and that the plurality of fans 17, plurality of vents 19, and plurality of lights 23 may be disposed anywhere on or within the kiosk 100.

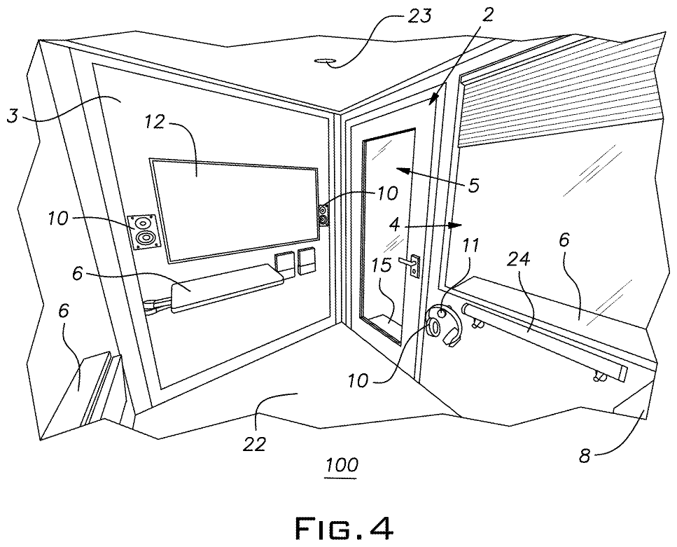

FIG. 4 illustrates an interactive modular hearing services soundproof kiosk 100. Although FIG. 4 illustrates single instances of the components, kiosk 100 may include any number of components. The kiosk 100 of FIG. 4 may reference and incorporate any components of kiosk of FIGS. 1-3. As shown in FIG. 4, the kiosk 100 includes a fitting room that includes an interior area within the kiosk 100 and after entry of first door 2 where the user is situated on seat bench 8.

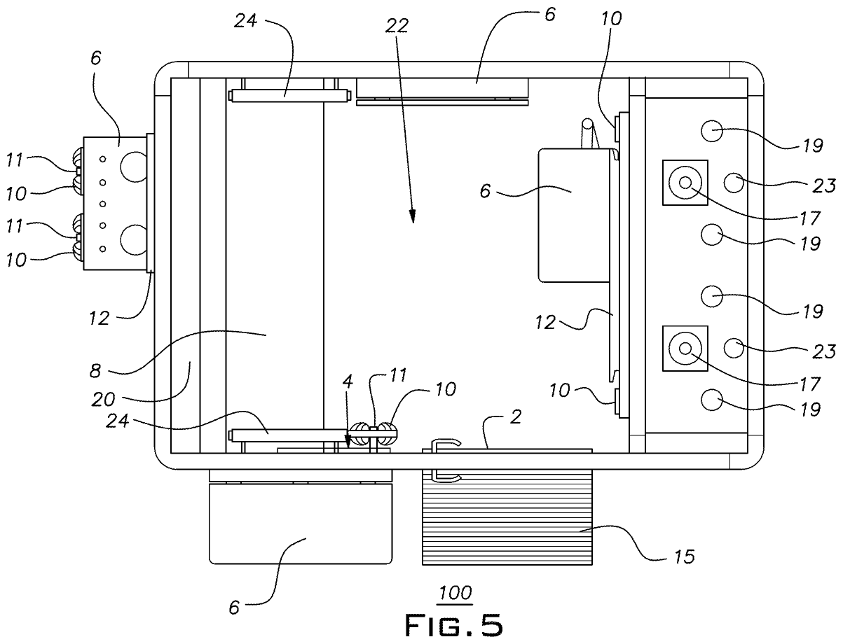

FIG. 5 illustrates a plan view of an interactive modular hearing services soundproof kiosk 100. Although FIG. 5 illustrates single instances of the components, kiosk 100 may include any number of components. The kiosk 100 of FIG. 5 may reference and incorporate any components of kiosk of FIGS. 1-4. As shown in FIG. 5, dimensions of the kiosk 100 are configured such that a distance in the x-direction of the outermost end (first end) of kiosk 100 to the other outermost end (second end) of kiosk 100 (exclusive of the shelf 6) may measure a distance of 108 inches, a distance in the y-direction of the outermost end (third end) of kiosk 100 to the other outermost end (fourth end) of kiosk 100 may measure a distance of 72 inches, and a distance in the y-direction from the outermost end (fourth end) of kiosk 100 to the outermost end of shelf 6 and ramp 15 may include 18 inches. Further, a distance in the x-direction of the storage room 13 from one end (fifth end) to another end (sixth end) may measure a distance of 21 inches. In addition, a distance in the x-direction of the fitting room of the kiosk 100 from one end (seventh end) to another end (eighth end) may measure a distance of 75 inches. Further, the width of the storage room 13 may measure a distance of 21 inches. In addition, the width of the fitting room may measure a distance of 75 inches. It is understood that these distances are exemplary and not intended to be limiting in any manner.

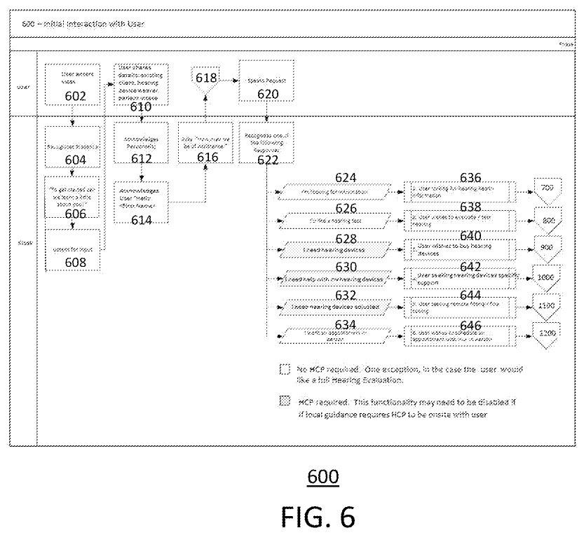

FIG. 6 illustrates a method 600 of initial interaction with a user using the interactive modular hearing services soundproof kiosk 100. FIG. 6 may reference and incorporate any components of kiosk of FIGS. 1-5. In addition, the kiosk 100 may include a memory and processor configured to carry out any of the functions performed by the kiosk 100. The processor may be a part of a device, such as a network-enabled computer.

As referred to herein, a network-enabled computer may include, but is not limited to a mobile computer device, or communications device including, e.g., a server, a network appliance, a personal computer, a workstation, a phone, a handheld PC, a personal digital assistant, a thin client, a fat client, an Internet browser, or other device. The device also may be a mobile device; for example, a mobile device may include an iPhone, iPod, iPad from Apple® or any other mobile device running Apple's iOS® operating system, any device running Microsoft's Windows® Mobile operating system, any device running Google's Android® operating system, and/or any other smartphone, tablet, or like wearable mobile device.

It is understood that the processing circuitry of the processor may contain additional components, including processors, memories, error and parity/CRC checkers, data encoders, anti-collision algorithms, controllers, command decoders, security primitives and tamper-proofing hardware, as necessary to perform the functions described herein. The device may further include a display and input devices. The display may be any type of device for presenting visual information such as a computer monitor, a flat panel display, and a mobile device screen, including liquid crystal displays, light-emitting diode displays, plasma panels, and cathode ray tube displays. Input devices may include any device for entering information into a user's device that is available and supported by the user's device, such as a touch-screen, keyboard, mouse, cursor-control device, touch-screen, microphone, digital camera, video recorder or camcorder. These devices may be used to enter information and interact with the software and other devices described herein.

The memory may be a read-only memory, write-once read-multiple memory or read/write memory, e.g., RAM, ROM, and EEPROM, and the kiosk 100 may include one or more of these memories. A read-only memory may be factory programmable as read-only or one-time programmable. One-time programmability provides the opportunity to write once then read many times. A write once/read-multiple memory may be programmed at a point in time after the memory chip has left the factory. Once the memory is programmed, it may not be rewritten, but it may be read many times. A read/write memory may be programmed and re-programed many times after leaving the factory. It may also be read many times.

The kiosk may include one or more networks. In some examples, a network may be one or more of a wireless network, a wired network or any combination of wireless network and wired network and may be configured to connect the device to a server. For example, the network may include one or more of a fiber optics network, a passive optical network, a cable network, an Internet network, a satellite network, a wireless local area network (LAN), a Global System for Mobile Communication, a Personal Communication Service, a Personal Area Network, Wireless Application Protocol, Multimedia Messaging Service, Enhanced Messaging Service, Short Message Service, Time Division Multiplexing based systems, Code Division Multiple Access based systems, D-AMPS, Wi-Fi, Fixed Wireless Data, IEEE 802.11b, 802.15.1, 802.11n and 802.11g, Bluetooth, NFC, Radio Frequency Identification (RFID), Wi-Fi, and/or the like.

In addition, the network may include, without limitation, telephone lines, fiber optics, IEEE Ethernet 902.3, a wide area network, a wireless personal area network, a LAN, or a global network such as the Internet. In addition, the network may support an Internet network, a wireless communication network, a cellular network, or the like, or any combination thereof. The network may further include one network, or any number of the exemplary types of networks mentioned above, operating as a stand-alone network or in cooperation with each other. The network may utilize one or more protocols of one or more network elements to which they are communicatively coupled. The network may translate to or from other protocols to one or more protocols of network devices. Although the network may be depicted as a single network, it should be appreciated that according to one or more examples, the network may comprise a plurality of interconnected networks, such as, for example, the Internet, a service provider's network, a cable television network, corporate networks, such as credit card association networks, and home networks.

The remote system associated with the health-care professional may include any number of servers. In some examples, the server may include one or more processors, which are coupled to memory. The server may be configured as a central system, server or platform to control and call various data at different times to execute a plurality of workflow actions. The server may be connected to the device via the network.

Any of the device, the remote system, and the kiosk may be configured to transmit and receive data to and from any of the components each constituting the device, the remote system, and the kiosk via the network.

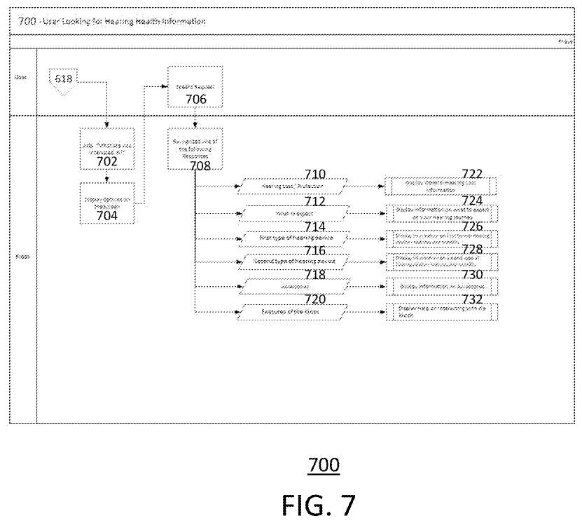

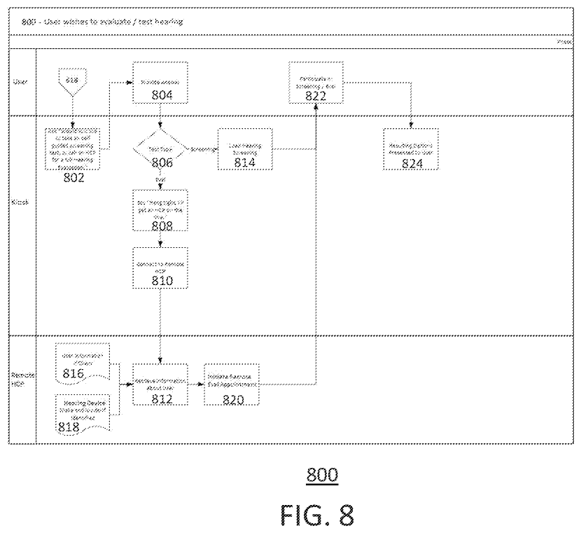

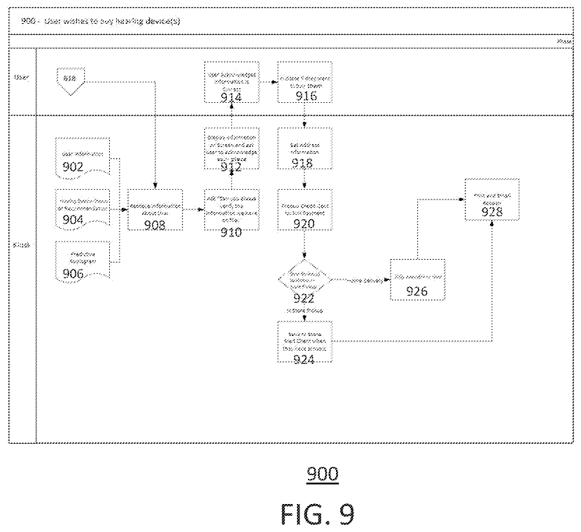

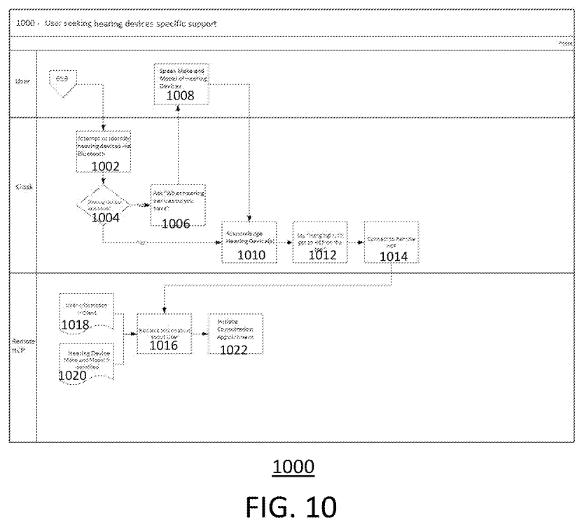

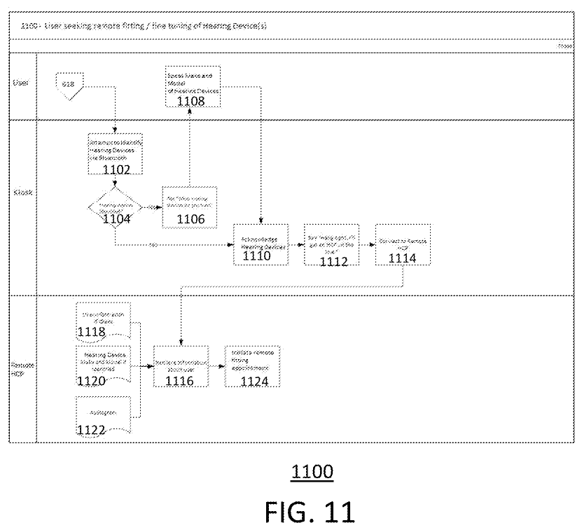

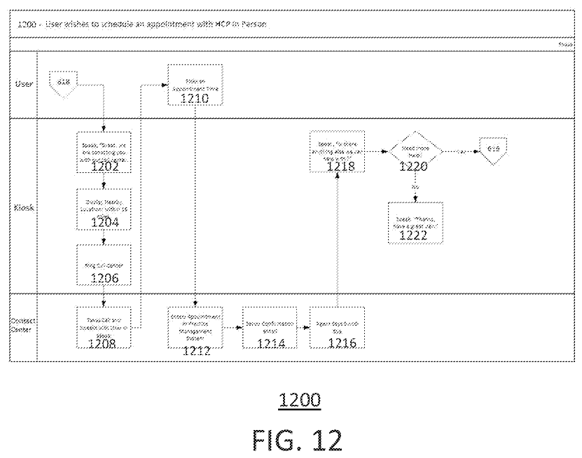

The methods of FIGS. 6-12 may be performed once the user is inside the kiosk 100, such as after the user has interacted with the outside screen relative to the kiosk and has thus decided to proceed to a more clinical evaluation, such as the second evaluation, by the inside screen of the kiosk. Referring back to FIG. 6, at block 602, the method 600 may include a user approaching and entering the kiosk 100. At block 604, the method 600 may include the kiosk 100 configured to recognize the presence of the user using motion sensors, proximity sensors, or the camera. At block 606, the method 600 may include the kiosk 100 configured to prompt one or more queries to the user. In some examples, the method 600 may include the kiosk 100 configured to provide one or more prompts including one or more queries to the user. For example, one of the queries of one or more prompts may include: “To get started, can we learn a little about you?” or the like. At block 608, the method 600 may include the kiosk 100 configured to wait for a response to the one or more prompts. At block 610, the method 600 may include the user providing one or more responses to the one or more prompts, such as whether they are an existing client, a wearer of one or more of the hearing device, the hearing instrument, the hearing aid, the hearing prosthesis, the hearing protection device, and/or the hearing system, and patient intake. At block 612, the method 600 may include the kiosk 100 configured to receive and acknowledge the one or more responses. At block 614, the method 600 may include the kiosk 100 configured to acknowledge the one or more responses by addressing the user by their first name. At block 616, the method 600 may include the kiosk 100 configured to ask the user how it can be of assistance. At block 618, the method 600 may include the user preparing to provide the request, and actually speaking the request at block 620. Based on the request, at block 622 the method 600 may include the kiosk 100 configured to recognize one of a plurality of responses, including but not limited to one of 624, 626, 628, 630, 632, and 634. Upon receipt of each of the recognized responses, the kiosk 100 may be instructed to interpret a response as being correspondent to a specific actionable instruction. At block 624, the kiosk 100 may be configured to recognize a response “I'm looking for information” as corresponding to indication that the user is looking for additional information at block 636. Thereafter, upon recognition of the response by the kiosk 100 at block 636, the method 600 may proceed to method 700 as explained below regarding FIG. 7. At block 626, the kiosk 100 may be configured to recognize a response “I'd like a hearing test” as corresponding to an indication that the user desires to evaluate and test hearing at block 638. Thereafter, upon recognition of the response by the kiosk 100 at block 638, the method 600 may proceed to method 800 as explained below regarding FIG. 8. At block 628, the kiosk 100 may be configured to recognize a response “I need hearing devices” as corresponding to an indication that the user desires to purchase one or more of the hearing device, the hearing instrument, the hearing aid, the hearing prosthesis, the hearing protection device, and/or the hearing system at block 640. Thereafter, upon recognition of the response by the kiosk 100 at block 640, the method 600 may proceed to method 900 as explained below regarding FIG. 9. At block 630, the kiosk 100 may be configured to recognize a response “I need help with my hearing devices” as corresponding to an indication that the user is looking for support specifically regarding one or more of the hearing device, the hearing instrument, the hearing aid, the hearing prosthesis, the hearing protection device, and/or the hearing system at block 642. Thereafter, upon recognition of the response by the kiosk 100 at block 642, the method 600 may proceed to method 1000 as explained below regarding FIG. 10. At block 632, the kiosk 100 may be configured to recognize a response “I need hearing devices adjusted” as corresponding to an indication that the user is seeking remote fitting and fine tuning of one or more of the hearing device, the hearing instrument, the hearing aid, the hearing prosthesis, the hearing protection device, and/or the hearing system at block 644. Thereafter, upon recognition of the response by the kiosk 100 at block 644, the method 600 may proceed to method 1100 as explained below regarding FIG. 11. At block 634, the kiosk 100 may be configured to recognize a response “I want an appointment in person” as corresponding to an indication that the user desires to schedule an appointment with a health-care professional (HCP) in person at block 646. Thereafter, upon recognition of the response by the kiosk 100 at block 646, the method 600 may proceed to method 1200 as explained below regarding FIG. 12. In some examples, responses 628, 630, and 632 may require a health-care professional prior to further action being taken by the kiosk 100, but may be disabled depending on local guidance. In addition, should the response by the user be construed as requiring a full hearing evaluation, then a health-care professional would be required prior to further action taken by the kiosk 100.

FIG. 7 illustrates a method 700 of interaction with a user using the interactive modular hearing services soundproof kiosk 100. FIG. 7 may reference and incorporate any components of kiosk of FIGS. 1-6. In particular, method 700 includes interaction when a user is seeking hearing health information. Without limitation, the method 700 may be responsive to user providing a request, such as from block 618, and in some examples, responsive to a response in block 624 and recognized by kiosk 100 as indicated in block 636.

At block 702, the kiosk 100 may be configured to ask the user what they are interested in. At block 704, the kiosk 100 may be configured to display a plurality of options on any of the one or more user display screens 12. At block 706, the user may provide a response by speaking their request to select one of the plurality of options. At block 708, the kiosk 100 may be configured to recognize the response as corresponding to one of the following responses, such as 710, 712, 714, 716, 718 before undertaking corresponding action. At block 710, the kiosk 100 may be configured to recognize a response of hearing loss and protection as corresponding to an indication that the kiosk 100 is configured to display general hearing loss information at block 722. At block 712, the kiosk 100 may be configured to recognize a response of what to expect as corresponding to an indication that the kiosk 100 is configured to display what to expect for the hearing journey associated with the user at block 724. At block 714, the kiosk 100 may be configured to recognize a response of a first type of one or more of the hearing device, the hearing instrument, the hearing aid, the hearing prosthesis, the hearing protection device, and/or the hearing system as corresponding to an indication that the kiosk 100 is configured to display specific information regarding the requested first type of one or more of the hearing device, the hearing instrument, the hearing aid, the hearing prosthesis, the hearing protection device, and/or the hearing system including features and benefits at block 726. At block 716, the kiosk 100 may be configured to recognize a response of a second type of one or more of the hearing device, the hearing instrument, the hearing aid, the hearing prosthesis, the hearing protection device, and/or the hearing system as corresponding to an indication that the kiosk 100 is configured to display specific information regarding the requested second type of one or more of the hearing device, the hearing instrument, the hearing aid, the hearing prosthesis, the hearing protection device, and/or the hearing system including features and benefits at block 728. At block 718, the kiosk 100 may be configured to recognize a response of accessories as corresponding to an indication that the kiosk 100 is configured to display information regarding the requested accessories at block 730. At block 720, the kiosk 100 may be configured to recognize a response of features of the kiosk 100 as corresponding to an indication that the kiosk 100 is configured to display help on interaction with the kiosk 100 at block 732. It is understood that the options set forth in blocks 710, 712, 714, 716, 718, and 720 are exemplary and not exhaustive, and thus do not represent the complete list of information available to the user.

FIG. 8 illustrates a method 800 of interaction with a user using the interactive modular hearing services soundproof kiosk 100. FIG. 8 may reference and incorporate any components of kiosk of FIGS. 1-7. In particular, method 800 includes interaction when a user wishes to evaluate testing and hearing. Without limitation, the method 800 may be responsive to user providing a request, such as from block 618, and in some examples, responsive to a response in block 626 and recognized by kiosk 100 as indicated in block 638.

At block 802, the kiosk 100 may be configured to inquire whether the user is interested in undertaking a self-guided screening test or call a health-care professional for a full hearing evaluation. At block 804, the user may provide the response to the query of block 802. At block 806, the kiosk 100 may be configured to receive the response so as to then begin the user-selected test type. At block 808, the kiosk 100 may be configured to inform the user that a health-care professional is being dialed up as part of the full hearing evaluation, in which the kiosk 100 may be configured to then connect remotely to a system, such as a computer, associated with the remote health-care professional (block 810). At block 812, the remote system may be configured to retrieve information about the user, which may be assisted by receiving input information from if the user is a client, such as block 816, as well as the make, model, and/or type of one or more of the hearing device, the hearing instrument, the hearing aid, the hearing prosthesis, the hearing protection device, and/or the hearing system at block 818. Upon receipt of this information, at block 820 the remote system may be configured to initiate a hearing services remote evaluation appointment. Alternatively, at block 814 the kiosk 100 may be configured to load a hearing screening as part of the self-guided screening test selected by the user at block 804. At block 822, the user may participate in the screening test of block 814 or appointment of block 820. Upon completion, at block 824, the kiosk 100 may be configured to display one or more resulting options to the user. Without limitation, the one or more resulting options may include actionable responses, such as establishing communication with a remote system, either through video, audio, or any combination thereof, and thereby enable calling-in to a remote health-care professional for hearing health care.

FIG. 9 illustrates a method 900 of interaction with a user using the interactive modular hearing services soundproof kiosk 100. FIG. 9 may reference and incorporate any components of kiosk of FIGS. 1-8. In particular, method 900 includes interaction when a user desires to purchase one or more of the hearing device, the hearing instrument, the hearing aid, the hearing prosthesis, the hearing protection device, and/or the hearing system. Without limitation, the method 900 may be responsive to user providing a request, such as from block 618, and in some examples, responsive to a response in block 628 and recognized by kiosk 100 as indicated in block 640.

At blocks 902, 904, and 906, information including user information, choice or recommendation of one or more of the hearing device, the hearing instrument, the hearing aid, the hearing prosthesis, the hearing protection device, and/or the hearing system, and a predictive audiogram may be provided to the kiosk 100. At block 908, the kiosk 100 may be configured to retrieve information about the user prior to forming a query. At block 910, the kiosk 100 may be configured to query the user regarding verification of information on file for the user. At block 912, the kiosk 100 may be configured to display information and acknowledge various aspects as part of the verification of the information on file for the user. At block 914, the user may acknowledge that the information being asked for verification is correct. At block 916, the user may indicate whether they want to purchase the one or more of the hearing device, the hearing instrument, the hearing aid, the hearing prosthesis, the hearing protection device, and/or the hearing system. At block 918, the kiosk 100 may be configured to obtain address information for the user for purchasing the one or more of the hearing device, the hearing instrument, the hearing aid, the hearing prosthesis, the hearing protection device, and/or the hearing system. At block 920, the kiosk 100 may be configured to process payment for the one or more of the hearing device, the hearing instrument, the hearing aid, the hearing prosthesis, the hearing protection device, and/or the hearing system. At block 922, the kiosk 100 may be configured to ship the one or more of the hearing device, the hearing instrument, the hearing aid, the hearing prosthesis, the hearing protection device, and/or the hearing system to a designated store for pick up or home delivery. For in store pick up, at block 924 the kiosk 100 may be configured to send the one or more of the hearing device, the hearing instrument, the hearing aid, the hearing prosthesis, the hearing protection device, and/or the hearing system to the designated store and inform the user upon its arrival, such as via a phone call, an email, or the like. For home delivery, at block 926 the kiosk 100 may be configured to ship the one or more of the hearing device, the hearing instrument, the hearing aid, the hearing prosthesis, the hearing protection device, and/or the hearing system to the user at a designated home address. Under either option of home delivery or in store pick up, at block 928, the kiosk 100 may be configured to print and/or email a receipt reflecting purchase transaction of the one or more of the hearing device, the hearing instrument, the hearing aid, the hearing prosthesis, the hearing protection device, and/or the hearing system.

FIG. 10 illustrates a method 1000 of interaction with a user using the interactive modular hearing services soundproof kiosk 100. FIG. 10 may reference and incorporate any components of kiosk of FIGS. 1-9. In particular, method 1000 includes interaction when a user is seeking support for a specific one or more of the hearing device, the hearing instrument, the hearing aid, the hearing prosthesis, the hearing protection device, and/or the hearing system. Without limitation, the method 1000 may be responsive to user providing a request, such as from block 618, and in some examples, responsive to a response in block 630 and recognized by kiosk 100 as indicated in block 642.

At block 1002, the kiosk 100 may be configured to attempt to identify the one or more of the hearing device, the hearing instrument, the hearing aid, the hearing prosthesis, the hearing protection device, and/or the hearing system via one or more connection types, including but not limited to Bluetooth®, Wi-Fi®, RFID. At block 1004, if the one or more of the hearing device, the hearing instrument, the hearing aid, the hearing prosthesis, the hearing protection device, and/or the hearing system are identified, then the kiosk 100 may be configured to acknowledge the one or more of the hearing device, the hearing instrument, the hearing aid, the hearing prosthesis, the hearing protection device, and/or the hearing system at block 1010 before informing the user that a remote health-care professional is in the process of being dialed up at block 1012, followed by subsequent connection to a remote system associated with the health-care professional at block 1014. If the one or more of the hearing device, the hearing instrument, the hearing aid, the hearing prosthesis, the hearing protection device, and/or the hearing system are not identified, then at block 1006 the kiosk 100 may be configured to inquire what one or more of the hearing device, the hearing instrument, the hearing aid, the hearing prosthesis, the hearing protection device, and/or the hearing system the user has. At block 1008, the user provides the make and model of the one or more of the hearing device, the hearing instrument, the hearing aid, the hearing prosthesis, the hearing protection device, and/or the hearing system by speaking, and then reverting to block 1010.

After connection to the remote system has been established, information about the user is retrieved at block 1016 based on whether the user is a client at block 1018 and based make and model of the one or more of the hearing device, the hearing instrument, the hearing aid, the hearing prosthesis, the hearing protection device, and/or the hearing system is identified at block 1020. Subsequently, a hearing services consultation appointment may be initiated by the remote health system associated with the health-care professional at block 1022.

FIG. 11 illustrates a method 1100 of interaction with a user using the interactive modular hearing services soundproof kiosk 100. FIG. 11 may reference and incorporate any components of kiosk of FIGS. 1-10. In particular, method 1100 includes interaction when a user is seeking remote fitting and fine tuning of one or more of the hearing device, the hearing instrument, the hearing aid, the hearing prosthesis, the hearing protection device, and/or the hearing system. Without limitation, the method 1100 may be responsive to user providing a request, such as from block 618, and in some examples, responsive to a response in block 632 and recognized by kiosk 100 as indicated in block 644.

At block 1102, the kiosk 100 may be configured to attempt to identify the one or more of the hearing device, the hearing instrument, the hearing aid, the hearing prosthesis, the hearing protection device, and/or the hearing system via one or more connection types, including but not limited to Bluetooth®, Wi-Fi®, RFID. At block 1104, if the one or more of the hearing device, the hearing instrument, the hearing aid, the hearing prosthesis, the hearing protection device, and/or the hearing system are identified, then the kiosk 100 may be configured to acknowledge the one or more of the hearing device, the hearing instrument, the hearing aid, the hearing prosthesis, the hearing protection device, and/or the hearing system at block 1110 before informing the user that a remote health-care professional is in the process of being dialed up at block 1112, followed by subsequent connection to a remote system associated with the health-care professional at block 1114. If the one or more of the hearing device, the hearing instrument, the hearing aid, the hearing prosthesis, the hearing protection device, and/or the hearing system are not identified, then at block 1106 the kiosk 100 may be configured to inquire what one or more of the hearing device, the hearing instrument, the hearing aid, the hearing prosthesis, the hearing protection device, and/or the hearing system the user has. At block 1108, the user provides the make and model of the one or more of the hearing device, the hearing instrument, the hearing aid, the hearing prosthesis, the hearing protection device, and/or the hearing system by speaking, and then reverting to block 1110.

After connection to the remote system has been established, information about the user is retrieved at block 1116 based on whether the user is a client at block 1118, one or more of the hearing device, the hearing instrument, the hearing aid, the hearing prosthesis, the hearing protection device, and/or the hearing system is identified at block 1120, and audiogram at block 1122. Subsequently, a hearing services consultation appointment may be initiated by the remote health system associated with the health-care professional at block 1124.

FIG. 12 illustrates a method 1200 of interaction with a user using the interactive modular hearing services soundproof kiosk 100. FIG. 12 may reference and incorporate any components of kiosk of FIGS. 1-11. In particular, method 1200 includes interaction when a user is desires to schedule an appointment with a health-care professional in person. Without limitation, the method 1200 may be responsive to user providing a request, such as from block 618, and in some examples, responsive to a response in block 634 and recognized by kiosk 100 as indicated in block 646.

At block 1202, the kiosk 100 may be configured to inform the user that they are being connected with the call center. At block 1204, the kiosk 100 may be configured to display a predetermined number of locations within a designated radius, including but not limited to 15 miles. At block 1206, the kiosk 100 may be configured to call the call center. At block 1208, a call center agent, who may also be associated with a remote system, may receive the call and get connected to speak with the user in the kiosk 100. Upon discussion, at block 1210, the user may select an appointment time. At block 1212, the remote system may be configured to enter the designated appointment time in a practice management system. At block 1214, the remote system may be configured to transmit a confirmation email reflecting the appointment time before terminating the session with the user at block 1216. At block 1218, the kiosk 100 may be configured to further inquire whether the user would like any additional assistance. At block 1220, the kiosk 100 may be configured to ask “Need more help?”. At block 1222, if more help is needed, the kiosk 100 may be configured to return to the initial state of options as presented above with respect to FIG. 6. At block 1222, if no more help is needed, the kiosk 100 may be configured terminate the session with the user.

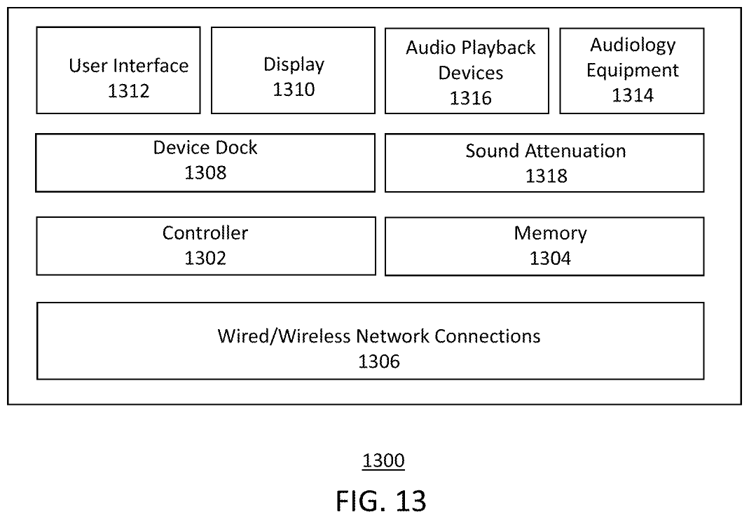

FIG. 13 illustrates a schematic diagram of a control system 1300. FIG. 13 may reference and incorporate any components of kiosk of FIGS. 1-12. Although FIG. 13 illustrates single instances of the components, control system 1300 may include any number of components. The control system 1300 may be part of the kiosk 100.

The control system 1300 may be configured to control any of the components, including but not limited to the one or more integrated media devices 10 and one more user display screens 12, of the kiosk 100. The control system 1300 may include a controller 1302, a memory 1304, wired/wireless connections 1306, a device dock (as a wired or wireless connection point) 1308, a display 1310, a user interface 1312, audiology equipment 1314, audio playback devices 1316, and sound attenuation 1318. In some examples, the control system 1300 may be external to the kiosk 100. In other examples, the control system 1300 may be internal to the kiosk 100. The controller 1302 may include one or more processors that are coupled to the memory 1304. The controller 1302 may be configured to communicate with user interface devices via user interface 1312. The user interface devices may include, for example, a touch-screen function of the one or more user display screens 12, push buttons, switches, joysticks, and/or any combination thereof. The controller 1302 may also configured to provide audio and/or video outputs to any number of the one or more media devices 10.

The wired/wireless network connections 1306 may comprise connections to a network (not shown) which may include telephone lines, fiber optic cables, a wide area network (WAN), a local area network (LAN), or a global network such as the Internet. In addition, the network may support a Global System for Mobile Communication (GSM), a Personal Communication Service (PCS), a Personal Area Network (PAN), Wireless Application Protocol (WAP), Multimedia Messaging Service (MMS), Enhanced Messaging Service (EMS), Short Message Service (SMS), Time Division Multiplexing (TDM) based systems, Code Division Multiple Access (CDMA) based systems, D-AMPS, Wi-Fi, Fixed Wireless Data, IEEE 802.11b, 802.15.1, 802.11n and 802.11g or any other wired or wireless network for transmitting and receiving a data signal.

The device dock 1308 may be configured for identification and programming of any number of one or more of the hearing device, the hearing instrument, the hearing aid, the hearing prosthesis, the hearing protection device, and/or the hearing system as previously explained.

The display 1310 may refer to any of the one or more user display screens 12, as previously explained. The user interface 1312 may be configured to communicate with the user interface devices via controller 1302.

The audiology equipment 1314 may comprise industry standard equipment used by a licensed hearing health provider, including but not limited to an audiometer, real-ear verification, and a video otoscope. Such equipment may be configured to provide hearing services by the remote hearing health provider, as previously explained.

The audio playback devices 1316 may comprise any number of devices that are configured to emit sound that can be measured and calibrated to industry standards for the purpose of simulating environments where hearing loss is most or frequently experienced.

The sound attenuation 1318 may refer to the materials specifically assembled and arranged within the kiosk 100 such that they are configured to limit the delivery of ambient sound into the interior space of the kiosk 100.

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology may be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one embodiment,” or “in one implementation” does not necessarily refer to the same example, embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An interactive hearing services kiosk, comprising:
one or more walls configured to define a space;
a first door providing access to the space;
one or more media devices;
one or more user display screens, at least one of the one or more user display screens is located on the outside of at least one of the one or more walls of the kiosk so as to define an outside display screen; and
a controller, wherein the controller is configured to:
display, via the outside display screen, a first set of one or more prompts that are associated with one or more hearing services;
receive a first input that is responsive to the first set of one or more prompts, the input including at least one response via the one or more media devices;
validate the first input; and
generate one or more recommendations based on a validated first input.

2. The interactive hearing services kiosk of claim 1, further comprising a user display screen that is located on the inside of the at least one of the one or more walls of the kiosk so as to define an inside display screen.

3. The interactive hearing services kiosk of claim 2, wherein the controller is configured to:
display, via the inside display screen, a second set of one or more prompts that are associated with one or more hearing services;
receive a second input that is responsive to the second set of one or more prompts, the second input including at least one response via the one or more media devices;
validate the second input;
generate one or more recommendations based on a validated second input; and
program one or more of a hearing device, a hearing instrument, a hearing aid, a hearing prosthesis, a hearing protection device, and/or a hearing system based on the one or more recommendations.

4. The interactive hearing services kiosk of claim 3, wherein the one or more media devices comprises one or more speakers that are configured to simulate one or more acoustic environments for a predetermined time duration, the one or more acoustic environments associated with a hearing evaluation to program one or more of a hearing device, a hearing instrument, a hearing aid, a hearing prosthesis, a hearing protection device, and/or a hearing system based on the second set of one or more prompts that are presented on the inside display screen.

5. The interactive hearing services kiosk of claim 2, wherein the controller is further configured to prompt a user for the first input responsive to a first evaluation that is presented on the outside display screen, and prompt the user for a second input responsive to a second evaluation that is presented on the inside display screen, the second evaluation including one or more queries than that belonging to the first evaluation.

6. The interactive hearing services kiosk of claim 3, wherein the controller is further configured to detect and identify the one or more of the hearing device, the hearing instrument, the hearing aid, the hearing prosthesis, the hearing protection device, and/or the hearing system via at least one selected from the group of near field communication, radio frequency identification, Bluetooth®, or Wi-Fi®.

7. The interactive hearing services kiosk of claim 3, wherein the controller is further configured to securely connect to a remote system and program the one or more of the hearing device, the hearing instrument, the hearing aid, the hearing prosthesis, the hearing protection device, and/or the hearing system based on received input from the remote system, the received input based on one or more hearing device configuration parameters of the one or more recommendations.

8. The interactive hearing services kiosk of claim 1, wherein the one or more media devices includes one or more selected from the group of a web camera, an audiometer, a video otoscope, a bone conduction transducer, a device configured for real-ear verification, an operator headphone, an operator microphone, a patient headphone, a patient microphone, an electronic signature pad, a patient response button, a push button interface, or a speaker.

9. The interactive hearing services kiosk of claim 3, wherein the controller is further configured to troubleshoot the one or more of the hearing device, the hearing instrument, the hearing aid, the hearing prosthesis, the hearing protection device, and/or the hearing system by controlling adjustment of one or more hearing device configuration parameters of the one or more recommendations.

10. The interactive hearing services kiosk of claim 1, wherein a user is configured to interact with the kiosk by at least one selected from a group of scanning of one or more QR codes, biometric input, haptic input, or a security code including a predetermined sequence of numbers, symbols, characters.

11. The interactive hearing services kiosk of claim 1, wherein the kiosk is configured to comply with a standard specifying a maximum permissible ambient noise level (MPANL) allowed for audiometric threshold assessment.

12. The interactive hearing services kiosk of claim 3, wherein the one or more recommendations comprise one or more hearing device configuration parameters that are automatically controlled by the controller, the one or more hearing device configuration parameters including a specific frequency gain of the one or more of the hearing device, the hearing instrument, the hearing aid, the hearing prosthesis, the hearing protection device, and/or the hearing system; feedback adjustments; a plurality of application programs configured to support improved hearing in noisy environments; tinnitus management capabilities of the one or more of the hearing device, the hearing instrument, the hearing aid, the hearing prosthesis, the hearing protection device, and/or the hearing system; or any combination thereof.

13. A method comprising:
generating, by a controller, a first set of one or more prompts that are associated with one or more hearing services;
instructing, by the controller, an outside display screen to display the first set of one or more prompts;
receiving, by the controller, a first input that is responsive to the first set of one or more prompts, the input including at least one response via one or more media devices;
validating, by the controller, the first input; and
generating, by the controller, one or more recommendations based on a validated first input.

14. The method of claim 13, wherein the one or more media devices comprises one or more speakers that are configured, by the controller, to simulate one or more acoustic environments for a predetermined time duration, the one or more acoustic environments associated with a hearing evaluation to program one or more of a hearing device, a hearing instrument, a hearing aid, a hearing prosthesis, a hearing protection device, and/or a hearing system based on a second set of one or more prompts that are presented on an inside display screen.

15. The method of claim 13, further comprising prompting, by the controller, a user for the first input responsive to a first evaluation that is presented on the outside display screen, and prompting, by the controller, the user for a second input responsive to a second evaluation that is presented on an inside display screen, the second evaluation including one or more queries than that belonging to the first evaluation.

16. The method of claim 13, wherein the one or more media devices includes one or more selected from the group of a web camera, an audiometer, a video otoscope, a bone conduction transducer, a device configured for real-ear verification, an operator headphone, an operator microphone, a patient headphone, a patient microphone, an electronic signature pad, a patient response button, a push button interface, or a speaker.

17. The method of claim 14, further comprising detecting, by the controller, and identifying the one or more of the hearing device, the hearing instrument, the hearing aid, the hearing prosthesis, the hearing protection device, and/or the hearing system at least one selected from the group of near field communication, radio frequency identification, Bluetooth®, or Wi-Fi®.

18. The method of claim 13, further comprising securely connecting, by the controller, to a remote system and programming, by the controller, one or more of a hearing device, a hearing instrument, a hearing aid, a hearing prosthesis, a hearing protection device, and/or a hearing system based on received input from the remote system, the received input based on one or more hearing device configuration parameters of the one or more recommendations.

19. A kiosk comprising the controller that is configured to perform the steps of claim 13.

20. A computer readable storage medium comprising computer program code instructions, being executable by a computer, for:
generating one or more prompts that are associated with one or more hearing services;
displaying the one or more prompts;
receiving input that is responsive to the one or more prompts, the input including at least one response via one or more media devices;
validating the input;
generating one or more recommendations based on a validated input.
